(19) United States
(12) Patent Application Publication
(10) Pub. No.: US 2005/0214759 A1
(43) Pub. Date: Sep. 29, 2005

(54) Enzymatic redox labelling of nucleic acids

(76) Inventors: Wjatschesslaw Wlassof, Belfast (GB); Garry Charles King, New South Wales (AU)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/500,171

(22) PCT Filed: Dec. 24, 2002
(86) PCT No.: PCT/AU02/01767

§ 371 Date: Dec. 22, 2004

(30) Foreign Application Priority Data
Dec. 24, 2001 (AU) PR 9752

(51) Int. Cl. C12Q 1/68; C07H 21/04

(52) U.S. Cl. 435/6; 536/25.32; 544/244; 544/243

(57) ABSTRACT

A modified nucleoside analogue having the formula (I): P-S-B-L-R where: P is a 5′ triphosphate or analogue or derivative thereof; S is a substituted or unsubstituted five- or six-membered sugar, sugar analogue or acyclo sugar analogue, but excluding a dideoxy-sugar, B is a substituted or unsubstituted nitrogenous base or base analogue or derivative thereof; L is a linker group; and R is a substituted or unsubstituted metallocene moiety or substituted or unsubstituted metal complex or substituted or unsubstituted redox-active organic moiety. The modified nucleoside is capable of enzymatic incorporation into a nucleotide chain and allows for redox labelling of nucleotides.

### FIELD OF THE INVENTION

The present invention relates to the synthesis, constitution and application of redox-tagged nucleoside analogues. More particularly, the present invention relates to nucleoside triphosphates for random or site-specific incorporation into nucleic acids by nucleotidyl transferases, especially template-dependent nucleotidyl transferases, along with the electrochemical detection of the resulting nucleic acid products.

### BACKGROUND OF THE INVENTION

Detection of specific nucleic acid sequences plays a central role in the identification of genes and in analysis of their expression and variation. The methods employed for these tasks can involve synthesis of nucleic acid probes by means of nucleotidyl transferase enzymes for the purposes of labelling or determination of base sequence identity. Labelling often involves the incorporation of a nucleotide which is chemically tagged or which is of a particular chemical composition so as to make it specifically detectable.

For many years, nucleotides and nuclei acids have been labelled with radioactive isotopes, most commonly ³²P. However, the use of the radioactive constructs carries a potential health risk and attendant regulatory complications, with additional inconvenience caused by radiolysis, short isotope half-lives and relatively cumbersome means of detection. In an early implementation of non-isotopic labelling, biotin-tagged nucleotides have previously been described. This application allowed efficient incorporation into DNA and RNA by the appropriate polymerases. Colourimetric detection of the label exploited the biotin-avidin interaction and an avidin-enzyme conjugate. More recently, hapten tagging methods such as digoxigenin-labelled (d)NTPs and antibody-enzyme conjugates have been introduced as an alternative. Biotin-, digoxigenin- and dinitrophenyl-nucleotides are in now widespread use. Currently, fluorescent tagging dominates applications in nucleic acid sequencing and microarray expression analysis. Fluorescent labelling offers increased sensitivity and the option for multicolour detection. In this as in other approaches, oligonucleotides can be labelled during chemical oligonucleotide synthesis, by incorporation of fluorescent-labelled nucleotides in the course of enzymatic synthesis or by post-synthetic derivatisation with a reactive dye construct. A broad variety of fluorescent-tagged NTPs, dNTPs, ddNTPs and acyclo-NTPs intended for enzymatic incorporation is now commercially available. In an elaboration of fluorescent methodology, nucleotides labelled with rare earth cryptates have recently been used to implement time-resolved fluorescence and FRET detection of nucleic acids.

Electrochemical detection (ECD) is the detection of molecules on the basis of the flow of electrons. Electrochemical detection offers a promising alternative to other approaches: it can be highly sensitive, rapid and amenable to inexpensive production in miniaturized (eg. lab-on-chip) formats. Several different implementations are currently being developed and commercialized. In one approach, unlabelled nucleic acids are detected with amol sensitivity by transition metal complex-mediated oxidation of guanine (G) nucleobases at potentials around 1.1 V.

Most other electrochemical implementations are based upon introducing one or more copies of a redox label, typically a metal complex, metallocene or quinone, by chemical conjugation. Due to its stability, ready synthetic access and ease of redox tuning, labelling with ferrocene has been the focus of significant attention. In early demonstrations of redox tagging, 5′-aminohexyl oligonucleotides were conjugated with ferrocene to enable electrochemical detection of hybridization and PCR products at fmol levels. Phosphoramidites of ferrocene for 5′ terminal labelling during oligonucleotide synthesis and 3′-end labelling of oligonucleotide have also recently been demonstrated.

For internal incorporation during chemical oligonucleotide synthesis, phosphoramidite monomers with a ferrocenyl moiety linked to position 5 of 2′-deoxyuridine and on-column derivatization of iodo-dU with ferrocenyl propargylarnide have been described, as have phosphoramidites labelled at the 2′-ribose position of adenosine and cytosine.

In addition to these approaches, non-specific internal labelling of DNA probes has been obtained by reaction with ferrocenecarboxaldehyde or aminoferrocene. The ability of a naphthalenediimide derivative of ferrocene to preferentially bind dsDNA via intercalation has been employed to detect hybridization.

The application of electrochemical methods to nucleic acids is not as advanced as fluorescence approaches. This field is developing with the construction of CE (capillary electrophoresis) chips with integrated ECD and the recent demonstration of a “four colour dye primer” analogue strategy for ECD DNA sequencing. However, the current art of preparing individual redox-labelled nucleic acids by phosphoramidite or post-synthesis reactions restricts the range of practical uses.

### SUMMARY OF THE INVENTION

The present invention provides a modified nucleoside analogue having a redox-label at the nitrogenous base.

The present specification discusses the constitution, synthesis and application of redox-tagged nucleoside analogues and more specifically NTPs for random or site-specific incorporation into nucleic acids, along with their electrochemical detection. Importantly, these analogues can be incorporated into oligo- and poly-nucleotides by a number of nucleotidyl transferases or polymerases (template-dependent nucleotidyl transferases) in the course of enzymatic synthesis. In some applications a high level of labelling can be achieved, allowing a significant increase in the sensitivity of detection.

Accordingly, in a first aspect the invention provides a modified nucleoside analogue having the formula (1):
P-S-B-L-R I
where:
P is a 5′ tri-phosphate or analogue or derivative thereof,
S is a substituted or unsubstituted five- or six-membered sugar, sugar analogue or acyclo sugar analogue, but excluding a dideoxy sugar;
B is a substituted or unsubstituted nitrogenous base or base analogue or derivative thereof;
L is a linker group; and
R is a substituted or unsubstituted metallocene moiety or substituted or unsubstituted metal complex or a substituted or unsubstituted redox-active organic moiety.

An advantage of these modified nucleoside analogues is that they are capable of enzymatic incorporation into a nucleotide chain.

In one embodiment, P is triphosphate or a triphosphate-containing moiety including α-, β-, or γ-thiotriphosphate, α-dithiotriphosphate, β,γ-methylenetriphosphate, or other enzyme-compatible triphosphate moiety. The nucleoside triphosphates of the present invention are most readily capable of being incorporated by enzymatic means into nucleic acid chains.

Preferably, group S is selected from substituted or unsubstituted ribose, 2′-deoxyribose, 3′-fluoro-2′-deoxyribose 3′-amino-2′-deoxyribose, a bicyclic “locked” LNA sugar such as 2′-O,4′-C-methylene-, 2′-C,4′-C-ethylene- or 2′-O,4′-C-ethylene-bridged furanose, or an acyclo moiety comprising a 2-hydroxyethoxymethyl group or analogue. Where the sugar is a substituted sugar, the substituent(s) may be one or more of fluoro, amino, hydroxyl, methyl, methoxy groups or other small substituents compatible with binding to the active site of nucleotidyl transferase enzymes.

Preferably, group B is a substituted or unsubstituted purine or pyrimidine or other nucleobase or nucleobase analogue. More preferably, B is an adenine, guanine, cytosine, uracil, or thymine derivative including the 7-deaza variants of adenine and guanine. If a nucleobase derivative is used, the nucleobase derivative preferably includes at least one thio or bromo group.

When the nucleobase is based on a purine structure, L is preferably attached to C8 of the purine structure. Where the base is the 7-deaza variant of the purine structure, L is preferably attached to C7 or C8. When the nucleobase is based upon a pyrimidine structure, L is preferably attached to C5. This provides a desirable orientation for L and R that extends away from an oligonucleotide following incorporation therein.

Preferably, L is a saturated or unsaturated aliphatic chain, with or without cyclic groups. Preferably L is 1-24 bonds in contour length, most preferably 3-12 bonds in length. L may include other groups, such as one or more amine groups. L preferably includes one or more carbon-to-carbon double or triple bonds to increase the rigidity of the linker. Most preferably, L is selected from propenyl or propylargyl derivatives, such as propenyl amine or propargyl amine.

R is a substituted or unsubstituted metallocene, a substituted or unsubstituted metal complex or an organic redox moiety. Suitable substituents include one or more of the groups fluoro, bromo, chloro, methyl, ethyl, hydroxy, hydroxymethyl, hydroxyethyl, methoxy, ethoxy, acetyl, cyano, thiocyano, amino, nitro, vinyl, amido, methylamido, and dimethylamido.

Preferably, suitable metallocenes include ferrocene and other metallocenes with redox potentials in the range of −1.0 to +1.0 V vs. standard hydrogen Electrode (SHE).

In one embodiment, the redox-active organic moiety is a quinone. Anthroquinone and substituted anthroquinones are especially preferred.

Preferably, suitable metal complexes include chelates and cryptates of transition metals such as iron, copper, cobalt, ruthenium and rhodium, osmium or other transition metals or non-transition elements with suitable redox behaviour. Suitable redox behaviour includes metal complexes exhibiting reversible electron transfer with Eo in the range of +1 V to −1 V vs. Standard Hydrogen Electrode.

A large number of ligands can be used to produce useful redox-labelled nucleosides.

Preferred ligands include tridentate ligands, especially those containing both 0 and N-metal donors. The metals include Fe, Ru and Os, as well as other metals, where two tridentate ligands will bind appropriately to the metal centre. By varying the metal centre as well as the properties of the ligands, the redox properties of the complexes will be tuned. The ligands include tridentate N-donor ligands, such as terpyridine (terpy), bis(benzimidazolyl)pyridines (bzimpy) and bis(pyrazolyl)pyridines (bpp), as well as mixed O,N,O donor ligands such as pyridinedicarboxylic acid (dipic) It will be appreciated that this list is not exhaustive and that the present invention extends to cover all ligands that produce suitable metal complexes in redox labelled nucleosides of formula I.

In a preferred embodiment, R is a substituted or unsubstituted metallocene moiety, more preferably a ferrocene.

In a second aspect, the invention provides a method of synthesising a modified nucleoside analogue according to the first aspect of the invention, the method comprising reacting a nucleoside or nucleotide precursor with a metallocene, metal complex or complexing agent or organic redox moiety precursor so as to form a link between the nucleos(t)ide analogue and the metallocene, metal complex or complexing agent or organic redox moiety. The method may further include the step of subsequently incorporating a 5′ triphosphate or derivative thereof if the starting nucleoside or nucleotide precursor does not include such a triphosphate or triphosphate derivative.

In a preferred embodiment, the link between the nucleos(t)ide precursor and the metallocene, metal complex or complexing agent or organic redox moiety is formed by a condensation reaction. In this embodiment, the method further includes the step of adding a condensing agent.

In another embodiment, the link between the nucleos(t)ide analogue and the metallocene, metal complex or complexing or organic redox moiety is formed by a displacement reaction.

In a preferred embodiment, the invention provides a method of synthesizing a modified nucleoside analogue according to the first aspect of the invention, the method comprising reacting a nuceloside or nucleotide precursor with a metallocene precursor in the presence of a condensing agent so as to form a link between the nucleoside analogue and the metallocene or derivative thereof.

In one embodiment the nucleotide precursor is 5-aminoallyl-uridine-5′-triphosphate, 5-aminopropargyl-uridine-5′-triphosphate, 5-aminoallyl-cytidine-5′-triphosphate, 5-aminopropargyl-cytidine-5′-triphosphate, 7-aminopropargyl-deazaadenosine-5′-triphosphate, 7-aminopropargyl-deazaguanosine-5′-triphosphate, 5-aminoallyl-2′-deoxyuridine-5′-triphosphate, 5-aminopropargyl-2′-deoxyuridine-5′-triphosphate, 5-aminoallyl-2′deoxycytidine-5′-triphosphate, 5-aminopropargyl-2′-deoxycytidine-5′-triphosphate, 7-aminopropargyl-7-deaza-2′deoxyadenosine-5′-triphosphate, 7-aminopropargyl-7-deaza-2′-deoxyguanosine-5′-triphosphate, 5-aminopropargyl-acyclouridine-triphosphate, 5-aminopropargyl-acyclocytidine-triphosphate, 7-aminopropargyl-acyclodeazaadenosine-triphosphate, or 7-aminopropargyl-acyclodeazaguanosine-triphosphate. This list is not exhaustive and other nucleotide precursors may also be used.

In one embodiment the metallocene precursor is a carboxylic acid. Preferably, the metallocene precursor is ferrocenecarboxylic acid or ferroceneacetic acid or derivative thereof.

Preferably, the condensing agent is selected from any one of a carbodiimide, for example dicyclohexylcarbodiimide, uronium compounds, activated esters and other compounds employed in the formation of amide bonds. Suitable condensing agents may include dicyclohexylcarbodiimide (DCC), 1-hydroxybenzotriazole (HOBT), succinimide esters, O-benzotriazol-1-yl-N,N,N′,N′-tetramethyluronium hexafluorophosphate (HBTU), N,N-diisopropylethylamine (DIPEA) or combinations of these agents.

In a preferred embodiment the condensing agent is O-benzotriazol-1-yl-N,N,N′,N′-tetramethyluronium hexafluorophosphate (HBTU).

In a third aspect, the present invention provides an oligo- or poly-nucleotide probe, primer or enzymatic reaction product comprising at least one residue of a nucleoside analogue according to the first aspect. Preferably, the at least one residue of a nucleoside analogue comprises at least one residue of a metallocene nucleoside analogue according to the first aspect of the invention.

In a fourth aspect, the present invention provides a method of nucleotide chain incorporation, the method comprising reacting a template nucleotide chain with a modified nucleoside analogue according to the first aspect in the presence of a processive nucleotidyl transferase or polymerase.

In a fifth aspect, the present invention is directed to a method of nucleotide chain extension, the method comprising reacting a nucleotide chain with a modified nucleoside analogue according to the first aspect in the presence of a non-processive nucleotidyl transferase such a terminal transferase or poly(A) polymerase.

Preferably, the modified nucleoside analogue is a triphosphate.

In a sixth aspect, the present invention provides a method of electrochemical detection of DNA, RNA, DNA/RNA chimers or nucleic acid analogues, the method comprising incorporating a modified nucleoside analogue according to the first aspect of the invention into a nucleic acid chain and detecting the analogue on the basis of its redox potential.

In a seventh aspect, the present invention provides a method of electrochemical detection of DNA, RNA, DNA/RNA chimers or nucleic acid analogues, the method comprising incorporating two or more different modified nucleoside analogues according to the first aspect of the invention, into the same or different nucleic acid chains, and detecting the modified nucleoside analogues on the basis of their different redox potentials.

The invention will hereinafter be further described by way of the following non-limiting examples and accompanying figures.

### BRIEF DESCRIPTION OF THE DRAWINGS

FIG. 1. Synthesis of ferrocene-labelled derivatives of UTP and dUTP.

FIG. 2. Cyclic voltammogram of Fc-dUTP.

FIG. 3. A. Structure of template-primer used for enzymatic incorporation of Fc-dUTP into DNA. B. Incorporation of Fc-dUTP into DNA by Klenow fragment and T4 DNA polymerase. The primer-template DNA of FIG. 3A was incubated with DNA polymerase and different sets of dNTPs (indicated at the top of the figure). The length of DNA fragments is shown on the left.

FIG. 4: Electrochemical detection of 60 fmol Fc-dU-labelled DNA following HPLC. Lower panel: UV detection at 260 nm. Upper panel: ECD at 700 mV.

FIG. 5: Alternative synthetic scheme for preparation of redox-labelled acyclouridine triphosphate.

FIG. 6: Second alternative synthetic scheme for preparation of redox-labelled acyclouridine triphosphate.

### DETAILED DESCRIPTION OF THE INVENTION

Unless defined otherwise, all technical and scientific terms used herein have the same meaning as commonly understood by one of ordinary skill in the art (eg., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2^{nd} ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, which are incorporated herein by reference) and chemical methods.

In a first aspect the invention provides a modified nucleoside analogue having the formula (1):
I: P-S-B-L-R I

where

P is a tri-phosphate or analogue or derivative thereof;

S is a substituted or unsubstituted five- or six-membered sugar, sugar analogue or acyclo sugar analogue, but excluding a dideoxy sugar;

B is a substituted or unsubstituted nitrogenous base or base analogue or derivative thereof;

L is a linker group, and

R is a substituted or unsubstituted metallocene moiety or substituted or unsubstituted metal complex or substituted or unsubstituted redox-active organic moiety.

Importantly, the modified nucleoside analogue of the present invention is capable of enzymatic incorporation into a nucleotide chain. Where the sugar is a 5- or 6-membered ring sugar, the modified nucleoside analogue of the present invention is capable of enzymatic incorporation into a nucleotide chain whilst allowing continuing chain growth to occur.

A nucleoside analogue is a compound which is capable of being incorporated by enzymatic or chemical means into a nucleic acid (DNA or RNA or chimeric DNA/RNA) chain, and is there capable of base stacking into the chain and base pairing or otherwise sterically accommodating a nucleotide residue in a complementary chain.

A natural nucleotide consists of a nitrogenous base, a sugar, and one or more phosphate groups. In a more general definition, a nucleotide analogue may include highly unnatural forms of these moieties, including extreme truncation of the sugar.

In the embodiment of this invention, group P is most commonly a triphosphate, or α-thio-triphosphate, but may include β- and γ-thiotriphosphates and other analogues that are enzyme-compatible moieties.

In both nucleosides and nucleotides the nitrogenous base is a purine or pyrimidine derivative. The two major purines are adenine and guanine, and the three major pyrimidines are cytosine, uracil, and thymine. The nitrogenous base may be modified. For example, for uridine the C4 substituent (O) may be replaced by S to form 4-thiouridine. For cytosine, H5 may be replaced by a methyl group to form 5-methylcytosine. In a 7-deaza purine derivative the N7 may be replaced by a C7. It is envisaged that further modifications could be made to the nucleoside derivative such that the nitrogenous base is replaced with an alternative aromatic group, for example a pyrrole or indole ring structure. Such modifications are included within the scope of the invention.

According to the present invention, the sugar structure of formulae I may be substituted or unsubstituted pentose or hexose or an acyclo moiety. Preferably, the pentose is a ribose, 2′-deoxyribose, 3′-fluororibose, 3′-aminoribose, 3′-fluoro-2′-deoxyribose, 3′-amino-2′-deoxyribose or 3′-azido-derivatives. Acyclo sugar replacements will also function as nucleotidyl transferase substrates.

R is a substituted or unsubstituted metallocene, a substituted or unsubstituted metal complex or an organic redox moiety. In one embodiment, suitable metallocenes include ferrocene and other metallocenes with redox potentials in the range of −1.0 to +1.0 V vs. standard hydrogen electrode (SHE). In an alternate embodiment, suitable metal complexes include chelates and cryptates of transition metals such as iron, copper, ruthenium and rhodium, or other non-transition elements with suitable redox behaviour.

Preferably, R is unsubstituted or substituted ferrocene. Various substituents may be selected to modify the redox potential of the ferrocene nucleoside analogues thereby providing different labels. Suitable substituents include nitro groups, primary, secondary and tertiary amines, hydroxy, alkoxy, amidate, halogen, alkyl and alkyl derivates and a range of other substituents compatible with substitution at the cyclopendadienyl ring. The redox-modifying substituents may be added to the ring of the ferrocene which is not attached to the linker group. This selectivity is caused by the electronic properties of the prior substituted ring, which directs substitution to the other ring.

Where R is a substituted metal chelate the metal ligands may also be selected to modify the redox potential of the metal chelate nucleoside analogue. This may be achieved by variation of donor atoms between oxygen, nitrogen, sulphur and other donors and by variation of ligand framework structure. As an alternative, the metal component of a single chelate or cryptate ligand may be varied to provide a range of redox potentials

Group R is linked to the nucleoside by a linker group L. The linker group is preferably a saturated or unsaturated aliphatic chain, with or without cyclic groups, preferably 1-24 bonds in contour length, most preferably 3-12 bonds in length. The degree of saturation may be varied. A higher proportion of double and/or triple bonds and/or aromatic rings gives greater rigidity. The carbon chain may be substituted with one or more nitrogen, sulphur and/or oxygen atoms. A wide range of linkage chemistries are compatible.

In a preferred embodiment, the linkage occurs via an alkyl amido group.

In a preferred embodiment of the second aspect, the invention provides a method of synthesising a modified nucleoside analogue according to the first aspect of the invention, the method comprising reacting a nucleoside or nucleotide precursor with a metallocene, metal chelate or organic redox moiety precursor in the presence of a condensing agent so as to form a link between the nucleio(s/t)ide analogue and the metallocene, metal chelate or organic redox moiety.

In a preferred embodiment, the invention provides a method of synthesising a modified nucleoside analogue according to the first aspect of the invention, the method comprising reacting a nucleoside or nucleotide precursor with a metallocene precursor in the presence of a condensing agent so as to form a link between the nucleoside analogue and the metallocene.

Nucleo(s/t)ide precursors can have a variety of forms, including derivatized nucleosides and mononucleotides. The preferred reaction involves a nucleoside triphosphate and a minimum number of chemical steps. A person skilled in the art can accomplish this synthesis by a number of methods.

Preferably the metallocene precursor is a metallocene carboxylic acid. In other embodiments, the metallocene precursor can also be another reactive form containing aklylamino, aldehyde, halogenated or other moieties. Preferably, when the modified nucleoside analogue is a ferrocene nucleoside analogue the metallocene precursor is ferrocenecarboxylic acid or ferroceneacetic acid.

Condensing agents are well known in the art and include dicyclohexylcarbodiimide and other carbodiimides in addition to uranium compounds, activated ethers and other compounds employed in the formation of amide bonds. In one embodiment the condensing agent is O-benzotriazol-1-yl-N,N,N′,N′-tetramethyluronium hexafluorophosphate (HBTU).

The nucleoside analogues of the present invention are useful for labelling DNA, RNA and DNA/RNA chimers or for incorporating into oligonucleotides.

In one aspect, the present invention is directed to an oligo-or poly-nucleotide probe, primer or other enzymatic reaction product comprising at least one residue of a metallocene nucleoside analogue according to the first aspect.

According to the present invention molecular probes or primers may be generated by recombinant or synthetic means. Generally the probe or primer is a polynucleotide that hybridises specifically to a target sequence. primers include for example a PCR primer or a primer for an alternate application reaction.

Generally, enzymatic reaction products include any products produced by an enzymatic reaction, such as by a polymerase reaction.

In another aspect, the present invention provides a method of nucleotide chain extension, the method comprising reacting a template nucleotide chain with a modified nucleoside analogue according to the first aspect in the presence of a processive nucleotidyl transferase or polymerase.

In a further aspect, the present invention is directed to a method of nucleotide chain extension, the method comprising reacting a nucleotide chain with a modified nucleoside analogue according to the first aspect in the presence of a non-processive nucleotidyl transferase such as terminal transferase or poly(A) polymerase.

Generally, a processive nucleitidyl transferase is a transferase which uses a template to polymerase nucleiotides into a complementary chain. A non-processive nucleotidyl transferase is one which is usually template-independent which produces a chain having a limited number of nucleotides.

Preferably, the modified nucleoside analogue is a nucleoside triphosphate.

The inventors present the first redox-tagged nucleoside triphosphates for labelling nucleic acids by common DNA and RNA polymerases with a view to facilitating the preparation of electrochemically-detectable nucleic acid probes.

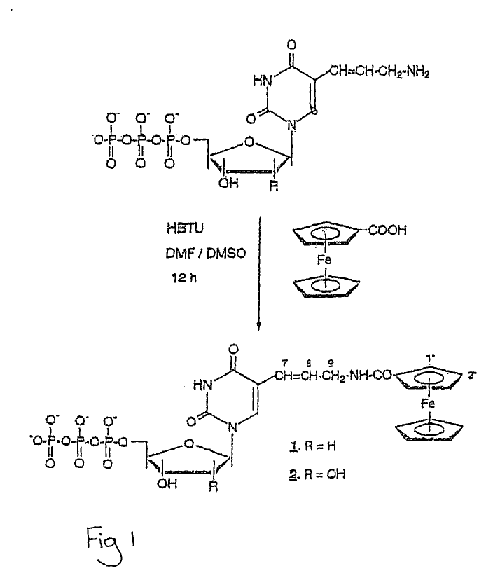

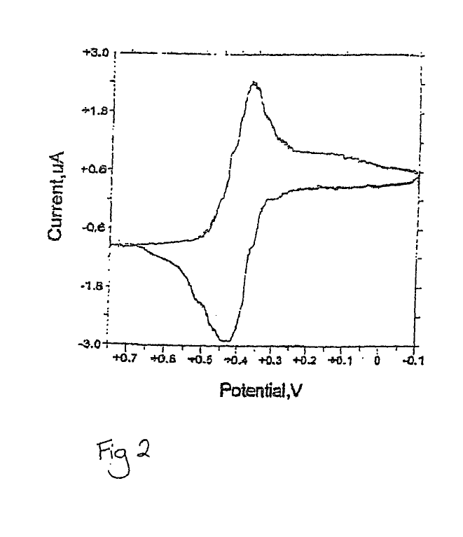

The ferrocene-labelled derivates of the present invention proved to be good substrates for commonly used polymerases, thus allowing a high degree of labelling. In one embodiment the inventors have demonstrated the synthesis of derivatives modified at position C5 of the pyrimidine ring (FIG. 1) using nucleoside triphosphates. The C5 modification rarely interferes with incorporation of modified nucleotides into DNA or RNA by the majority of polymerases. Even dUTP and UTP derivatives with bulky C5 substituents can be successfully used as substrates for these enzymes. It is understood that the substrate qualify of any particular nucleotide derivative will vary between polymerases.

In one aspect, the present invention provides a method of electrochemical detection of DNA, RNA, DNA/RNA chimers or nucleic acid analogues, the method comprising incorporating a modified nucleoside analogue according to the present invention into a nucleic acid chain and detecting the analogue on the basis of its redox potential.

In another aspect, the present invention provides a method of detection of DNA, RNA, DNA/RNA chimers or nucleic acid analogues, the method comprising incorporating two or more different modified nucleoside analogues according to the present invention into the same or different nucleic acid chains, and detecting the modified nucleoside analogues on the basis of their different redox potentials. This involves the production of redox-labelled nucleotides with different redox potentials, incorporation of these nucleotides into nucleic acid, followed by simultaneous detection and quantification.

In one embodiment, labelling one type of nucleotide (eg dUTP) with two different redox tags, followed by incorporation of these nucleotides separately into cDNAs corresponding to different treatments, mixing of the RNAs and simultaneous detection renders an electrochemical analogue of two-colour mRNA expression analysis. When redox-labelled terminator nucleotides (usually those lacking a 3′OH group, or more generally those nucleotides that cause termination of enzymatic chain elongation following their incorporation into the chain) are employed such that nucleotides corresponding to each of the four common bases A, G, C and T carry different redox groups, an electrochemical analogue of four-colour dye-terminator nucleic acid sequencing will be enabled. In a similar embodiment, analysis of nucleic acid polymorphisma (SNPs and indels) by primer extention methods can be enabled.

A person skilled in the art of the present invention could provide the invention in a kit. The kit may contain components necessary to practice the invention. For example, a kit may contain a vial(s) of redox-labelled nucleotide(s), a vial of nucleotidyl transferase enzyme(s), an appropriate unlabelled nucleotide mix, an optimised reaction buffer, control template and primer so that the user may determine the efficiency of DNA synthesis. In this case, the user would apply specific primer and template nucleic acids for the application.

Electrochemical Detection

Electrochemical detection can be employed in liquid chromatography, capillary electrophoresis, microchannel electrophoresis (see Kissinger and Heineman, Laboratory techniques in Electroanalytical Chemistry, Dekker, N.Y., 1996) and in microarray formats. It has been demonstrated that electrochemical detection is very sensitive, being able to measure amol to zmol quantities of sample in n1 to p1 volumes. Electrochemical methods have been used to detect labelled DNA during BPLC (Johnston, 1995; Shigenaga, 1990; Takenaka et al., 1994), microcapillary electrophoresis (Woolley et al., 1998) and in a microarray format (Umek et al., 2001). In the Examples below HPLC-ECD has been used due to the local availability of instrumentation. The separation power of this method is low in comparison to CE, but is adequate for demonstration purposes.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### EXAMPLES OF THE INVENTION

The invention will now be described in connection with certain preferred embodiments in the following examples so that aspects thereof may be more fully understood and appreciated. It is understood that the examples are not intended to limit the invention to these particular embodiments.

Abbreviations:

DMF, dimethylformamide;

DMSO, dimethylsulfoxide;

DTT, dithiotreitol;

EDTA, ethylenediaminetetraacetic acid;

Fc-UTP, 5-(3ferrocenecarboxamidopropenyl-1)-uridine-5′-triphosphate;

Fc-dUTP, 5-(3-ferrocenecarboxamidopropenyl-1)-2′-deoxyuridine-5′-monophosphate;

HBTU, O-benzotriazol-1-yl-N,N,N′,N′-tetramethyluronium hexafluorophosphate;

PAGE, polyacrylamide gel elecvtrophoresis;

PCR, polymerase chain reaction;

RP HPLC, reverse phase high pressure liquid chromatography;

TEAB, triethylammonium bicarbonate;

### Materials and Methods for the Examples

Unless otherwise stated, starting materials for the chemical synthesis were obtained from Sigma-Aldrich or Bio-Rad and were used without further purification.

5-(3-aminopropenyl-1)-uridine-5′-triphosphate and 5-(3-aminopropenyl-1)-2′-deoxyuridine-5′-triphosphate were prepared according to the reported procedure (Langer et al., 1981).

Oligonucleotides were purchased from Sigma Genosys and purified by denaturing PAGE (20% acrylamide/8 M urea) as described (Sambrook et al., 1989).

The Klenow fragment of E. coli DNA polymerase I was purchased from NEB. T4 DNA polymerase was from MBA Fermentas. T7 RNA polymerase was from USB. Tth DNA polymerase was from Perkin Elmer. ¹H and ³¹P NMR spectra were recorded on a Bruker DMX-300 spectrometer. Chemical shifts are reported in parts per million (δ) relative to an external standard.

UV spectra and DNA melting experiments were performed on a Cary 100 Bio spectrophotometer (Varian). HPLC separation and analyses were performed with an Akta Purifier system (Pharmacia Biotech) monitored at 260 and 440 nm. A reverse phase C18 column (Zorbax ODS, 250-9.4 mm) was utilised for preparative separations.

PAGE was run using a Protean IIxi cell (Bio-Rad) with 20 cm glass plates. Gels were run at 600 V in 0.09 M tris-borate, 2 mM EDTA running buffer and stained with SYBR Green II (Molecular Probes) before scanning with a Fluor-S MultiImager (Bio-Rad). Agarose gels were run at 5 V/cm in a Gello-tank cell (HyBaid) in 0.0945 M tris-borate, 1 mM EDTA buffer.

HPLC analyses of ferrocene-labelled DNA samples with both optical and electrochemical detection were performed with a Shimadzu High Performance Liquid Chromatograph equipped with LC-10A Solvent Delivery Module, SIL-10A Auto Injector, DGU-14A degasser, SPD-M10A UV/VIS photodiode array detector, and ESA Coulochem II electrochemical detector (ESA, Inc.) (Guard Cell Model 5020 (potential—0.8 V), Standard Analytical Cell Model 5010 (potential—0.7 V)). A Vydac reverse phase Protein & Peptide C18 column (250×4 mm) was used for analyses.

### Example 1

### Synthesis of Fc-dUTP and Fc-UTP

A 45 μmol sample of 5-(trans-3-aminopropenyl-1) 2′-deoxyuridine 5′-triphosphate was evaporated twice from absolute ethanol to remove traces of water before dissolving in 1 ml anhydrous DMF. A solution of 23 mg (0.1 mmol) ferrocenecarboxylic acid in DMSO and 37.9 mg (0.1 mmol) solid HBTU were added to the nucleotide solution with stirring until dissolution of HBTU and the mixture incubated at room temperature overnight. The reaction mixture was diluted with 20 ml of 5 mM 2-mercaptoethanol in water and the yellow ferrocenecarboxylic acid precipitate removed with a 0.45 μm polypropylene membrane filter (Gelman Sciences). The filtrate was applied to a DEAE-cellulose column (1×25 cm) equilibrated with 5 mM aqueous 2-mercaptoethanol and separated with a linear gradient of TEAB (0-0.35 M, 500 ml) in 5 mM 2-mercaptoethanol. Product eluted as a large peak at the end of the gradient.

The product fractions were pooled, evaporated, and purified by RP HPLC with a linear gradient of acetonitrile (0-30%) in 0.05 M LiC10₄. Solvent was removed by rotary evaporation, the residue dissolved in 0.5 ml water and the product precipitated by addition of 5 ml 2% LiC10₄ in acetone. The precipitate was washed with acetone and dried on air. Fc-dUTP yield 14 μmol (30%). UV (H₂O) [symbol] ₘₐₓ=439 nm (ε=300 M⁻¹ cm⁻²). ¹H NMR (D₂O) δ 2.36 (m, H2′, 2H), 3.98 (d, J=4.5 Hz, H9, 2H), 4.19 (m, H4′, H5′, 3H), 4.27 (s, C₅H₅ of Fc, 5H), 4.51 (s, H2′, 2H), 4.77 (m, H3′, 1H), 4.81 (s, H1″, 2H), 6.27 (t, J=6 Hz, H1′, 1H), 6.39 (s, H7, 1H), 6.48 (t, J=4.5 Hz, H8, 1H), 7.88 (s, H6, 1H).

An identical procedure was used for the synthesis of Fc-UTP (yield 7%).

### Example 2

### Characterisation

Ferrocene-labelled dUTP (Fc-dUTP, 1) and UTP (Fc-UTP, 2) derivatives (FIG. 1) were successfully synthesized by reaction of the 5-(3-aminopropenyl)-nucleoside triphosphates with ferrocenecarboxylic acid in the presence of HBTU. This procedure generates a relatively rigid 6-bond linkage between the nucleobase and redox label. The products were purified to homogeneity by ion-exchange chromatography followed by RP HPLC. The yields of both products were relatively low (30% for Fc-dUTP and 7% for Fc-UTP), probably due to steric hindrance in the course of the reaction. We have also used this procedure to synthesize a dUTP derivative adducted to ferroceneacetic acid.

Fc-dUTP and Fc-UTP have characteristic absorption spectra which correspond to a superposition of spectra for the modified nucleotide and ferrocene carboxamide constituents. They have a strong absorption in the UV region and a seal, broad peak characteristics of ferrocene near 440 nm. Cyclic voltammetry of Fc-dUTP yields a symmetric peak with E_{1/2}=398 mV vs. Ag/AgCl, consistent with reversible redox reaction of the Fc moiety. The redox potential of Fc-dUTP is 90 mV greater than the potential of ferrocenecarboxylate (310 mV vs. Ag/AgCl) measured in the same buffer (data now shown), reflecting the change of pentadienyl ring substituent (—COO to —CONHR) to one which is more electron-withdrawing. The observed potential is close to that reported for a ferrocene carboxamide moiety attached to the 5′-end of DNA oligonucleotides in aqueous buffer (406-425 mV vs. Ag/AgCl).

### Example 3

### Cyclic Voltammetry

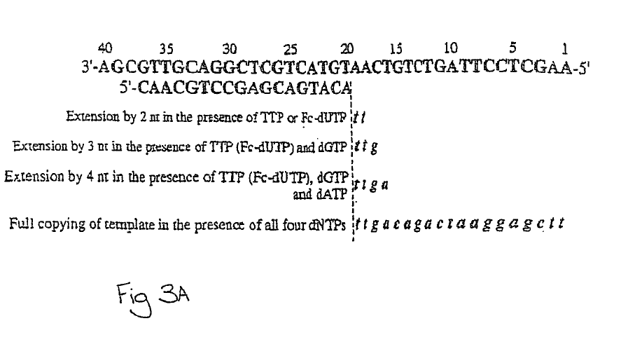

Cyclic voltammograms were recorded with an electrochemical analyser (BAS). The three-electrode system consisted of a glassy carbon working electrode, a Ag/AgCl (saturated KCl) reference electrode (E_{ref}=206 mV) and a platinum counter electrode. Experiments were performed in a 5 ml electrochemical cell containing 0.8 mM Fc-NTP in 20 mM tris-acetate (pH 7.4), 100 mM KCl, and 1 mM MgCl₂ at a scan rate of 20 mV/s. The scan range was from −0.1 to +0.8 V (vs. Ag/AgCl). (See FIG. 2).

### Example 4

### Primer Extension by DNA Polymerases

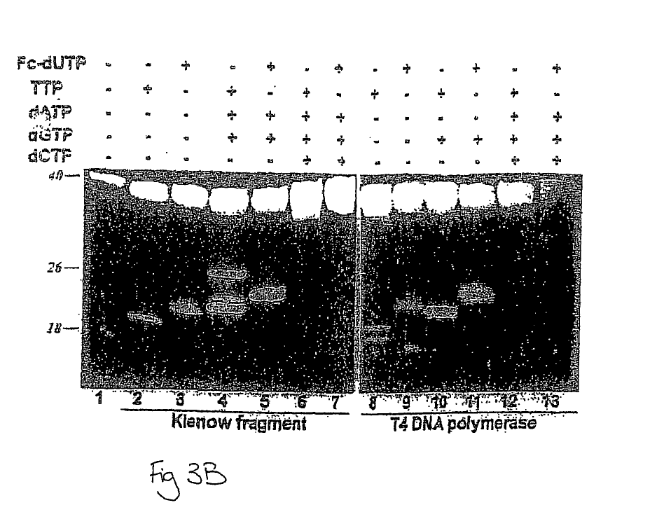

A DNA partial duplex consisting of an 18-mer primer 5′-5 CAACGTCCGAGCAGTACA and a 40-mer template 5′-AAGCTCCTTAGTCTGTCAATGTACTGCTCGGACGTTGCGA (FIG. 3A) was prepared by annealing PAGE-purified oligonucleotides. DNA duplex (2 μM) was incubated in 20 μl polymerase reaction mixture (6.7 mM tris-HCl pH 8.8, 6.6 mM MgCl₂, 1 mM DTT, 16.8 mM (NH₄)₂SO₄, 200 μM dNTPs and 0.25 U/μl Klenow fragment or T4 DNA polymerase) for 20 min at room temperature. Reactions were stopped with an equal volume of gel loading buffer (98% formamide, 10 mM EDTA pH 8.0, 0.025% bromophenol blue, 0.025% xylene cyapole FF), heated at 95° C. for 2 min and subjected to denaturing PAGE (see FIG. 3B). The substrate properties of Fc-dUTP were tested in DNA polymerase-catalysed primer extension assays using the model DNA duplex shown in FIG. 3A. The sequence of the template allows the progress of primer extensions to be controlled by omitting some dNTPs from the reaction mixture. The results of incubating primer-template with E. coli DNA polymerase I Klenow fragment or T4 DNA polymerase are shown in FIG. 3B. Addition of unlabelled dTTP to the reaction mix results in extension of the 18-mer primer (lane 1) by 2 nucleotides (lanes 2 and 8). The product heterogeneity displayed by T4 DNA polymerase (lane 8) is caused by its stronger 3′-5′ exonuclease activity, which is also evidence in lanes 9-11. When Fc-dUTP replaces dTTP, both DNA polymerases incorporate two consecutive Fc-dUMP residues into the 3′-end of the primer (lanes 3 and 9). Because the incorporated pFc-dU residue has a molecular weight almost twice that of the pT residue (574 vs. 321 Da) and the bulky adduct also alters the hydrodynamic properties of the chain, the mobility of the Fc-dUTP-extended primer is significantly lower than that of its natural counterpart. There is an indication that a small fraction of the primer is not extended by Klenow fragment (lane 3), but this behaviour is not consistent across the gel.

Some modified nucleoside triphosphates have the properties of terminators, their incorporation into DNA preventing or slowing further extension. To check this possibility, we incubated the primer-template with Fc-dUTP and one or two other dNTPs required for limited primer extension. In the presence of Fc-dUTP, dGTP and DATP, Klenow fragment successfully extends the chain following Fc-dU incorporation (lane 5). Similarly, T4 DNA polymerase extends the primer by three residues in the presence of Fc-dUTP and dGTP (lane 11). This allows us to conclude that Fc-dUTP is both efficiently incorporated and does not significantly inhibit further extension. Of some interest, Klenow fragment displays cleaner extension behaviour with the Fc-dUTP/dATP/dGTP mixture (lane 5) than with dTTP/dATP/dGTP (lane 4), where misincorporation at G15 has allowed the formation of a minor 26-mer product which terminates at the next “stop” position, G11. Incubation of the primer-template with all four natural dNTPs (lanes 6 and 12) or Fc-dUTP plus three dNTPs (lanes 7 and 13) allows run-off extension of the primer. Again no visible termination was registered when Fc-dUTP replaced dTTP.

### Examples 5

### DNA Labelling with Fc-dUTP in the Course of PCR

A segment of the T4 DNA ligase gene (positions 1001 to 1988) was use as a model sequence for amplification in the poresence of a ferrocene-labelled TTP analogue. The gene was cloned into plasmid pKL01. The 25-mer 5′-GCT GAT GGA GCT CGG TGT TTT GCT T-3′ was used as a forward primer, and 31-mer 5′-TAT ATA AGC TTC ATA GAC CAG TTA CCT CAT G-3′ was used as a reverse primer. The use of these primers allows formation of a 998 nt long amplicon. The reaction mixtures (20 uL each) contained 6.7 mM tris-HCl (pH 8.8), 1.66 mM (NH₄)₂SO₄, 0.045% Triton X-100, 0.02 mg/mL gelatin, 2.5 MM MgCl₂, 0.2 uM each primer, 20 ug/mL pKL01 plasmid, 0.2 mM dNTPs, and 0.1 U/uL Tth polymerase (exo). In some reaction mixtures, TTP was partially or fully substituted with Fc-dUTP in such a way that the total concentration of TTP and Fc-dUTP was still 0.2 mM. Conditions of PCR were as follows: 2 min at 95° C., and then 22 cycles at 94° C. for 30 sec, 50° C. for 30 sec, 50° C. for 1 min, and 70° C. for 10 min. After amplification, 4 uL of gel loading buffer (30% glycerol, 0.25% bromphenol blue and 0.25% xylene cyanole FF) was added, and samples were analysed on 1% agarose gel.

Full substitution of TTP by Fc-dUTP did not support the formation of a PCR product by Tth DNA polymerase. However, when TTP was substituted by Fc-dUTP at 25%, 50%, or 75%, synthesis of the correct amplicon was observed. The amplicon molecular showed a progressive increase in molecular weight with increasing Fc-dUTP:dTTP ratio, indicating extensive Fc-dUMP incorporation.

### Example 6

### Incorporation of Pc-UTP into RNA in the Course of Transcription

Circular plasmid pT7Mta which contains the promoter for T7 RNA polymerase followed by the gene for aptamer C40 and a T7 terminator sequence was used for transcription. T7 RNA polymerase tends to produce short abortive RNA transcripts when modified nucleotides are incorporated into the first 12 nucleotides of RNA. To avoid this potential complication, we used a template which does not contain A residues in the first 18 nucleotides of the coding sequence.

A typical transcription mixture (10 μL) contained 40 mM tris-HCl (pH 8.0), 15 mM MgCl₂, 5 mM DTT, 0.05 mg/mL BSA, 1 UOL Rnasin, 0.4 mM NTPs, 10 ug/mL pT7Mta template, and 10U/uL T7 RNA polymerase. In some reaction mixtures, UTP was partially or fully substituted with Pc-UTP in such a way that the total concentration of UTP and Fc-UTP was still 0.4 mM. Reaction mixtures were incubated at 37° C. for 2 h. Reactions were stopped by addition of 10 μL of gel loading buffer (98% formamide, 10 mM EDTA (pH 8.0), 0.025% bromophenol blue and 0.025% xylene cyanole PF) and heated at 95° C. for 2 min. RNA fragments were separated by 10% PAGE/8 M urea. The gel was stained by SYBR Green II (Sigma) according to manufacturers procedure, and visualised on a Fluorimager (Bio-Rad).

Due to inefficient T7 termination in this construct, a majority of the RNA products formed are significantly longer than the intended 117 nt long product. Nonetheless, large quantities of Fc-UMP-labelled RNA were produced. Substitution of UTP by Fc-UTP caused a significant decrease in the amount of RNA product formed. However, even in the total absence of UTP, T7 RNA polymerase synthesized a significant amount of RNA product.

### Example 7

### Electrochemical Detection of Labelled Polynucleotides During HPLC

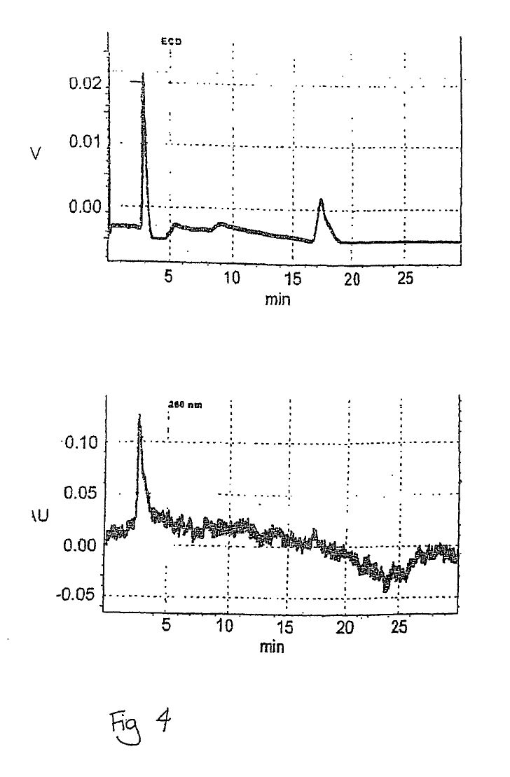

4 μM duplex DNA (40-mer template 5′-AAGCTCCTTAGTCTGTCAATGTACTGCT CGGACGTTGCTA-3′ and 18-mer primer 5′-CAACGTCCGAGCAGTACA-3′) was incubated in 240 μL of reaction mixture consisting of 6.7 mM tris-HCl (pH 8.8), 6.6 mM MgCl₂, 1 mM DTT, 16.8 mM (NH₄)₂SO₄, 200 μM dNTPs (except TTP), 200 μM Fc-dUTP, and 0.25 U/μL of Klenow fragment for 20 min at room temperature. Low molecular weight components were separated on Bio-Spin 30 chromatography column (Bio-Rad). The eluate was extracted with equal volumes of phenol/chloroform (1:1) and chlorophorm. DNA was precipitated by addition of 10 volumes of 2% LiC10₄ in acetone and centrifugation (12000 g, 15 min). Precipitate was dried in vacuo, redissolved in 200 μL of HPLC buffer (50 mM LiC10₄/2.5% acetonitrile in water), and the DNA concentration was determined spectrophotometrically by absorption at 260 nm. Different amounts of sample were loaded onto the analytical reverse-phase column (Vydac, Protein & Peptide C18, 250×4 mm) and analysed by isocratic elution with optical (260 nm) and electrochemical (E=0.7V) detections (flow rate—1 mL/min). After being extended in the presence of all 4 dNTPs including Fc-dUTP instead of TTP, the model DNA duplex would contain five Fc-dUMP residues. We used this ferrocene-labelled duplex for electrochemical detection in the course of RP HPLC. The HPLC system was equipped with both optical and electrochemical detectors as described in Materials and Methods. Different quantities of DNA duplex were injected on the reverse phase column and eluted in isocratic mode by 50 mM LiC10₄/2.5% acetonitrile in water. The eluate was monitored optically at 260 nm and electrochemically at 0.7 V. In our conditions, the retention time for DNA duplex was 17.5 min. Only picomolar quantities of DNA were reliably detected with UV/VIS photo array detector, while electrochemical detection allowed to register femtomolar amounts of the duplex (FIG. 4).

### Example 8

### Melting Analysis of DNA Duplexes Containing Fc-dUMP Residues

DNA samples for melting experiments were prepared as described above in the preparation of DNA duplex for electrochemical detection. As a control sample, unmodified DNA duplex containing all natural nucleotides was prepared using the same procedure. Both DNA duplexes were dissolved in 1 mL of 0.3 M KH₂PO₄ (pH 7.0) and transferred into standard quartz cuvettes. The melting curves were obtained by recording the changes in absorption of samples at 260 nm with increase of temperature from 25° C. to 95° C. (temperature gradient 1° C. per min).

Modification of natural components of nucleic acid can sometimes severely affect the stability of the DNA duplex. This issue is very important for all applications where formation of DNA hybrids is involved. To check the effect of incorporation of ferrocene-modified nucleotides into DNA, we have measured the melting temperature of a DNA duplex containing 5 residues of Fc-dUMP. The melting of unmodified duplex with the same sequence was studied for comparison. The melting temperature of modified DNA hybrid (71°) is only 4 degrees lower than the one of normal duplex (75° C.). This allows us to conclude that modification by ferrocene at the C5 position of dUMP does not significantly disrupt the native structure of DNA.

### Example 9

### Synthesis of Ferrocene-Labelled Acyclonucleotide Triphosphate Derivatives

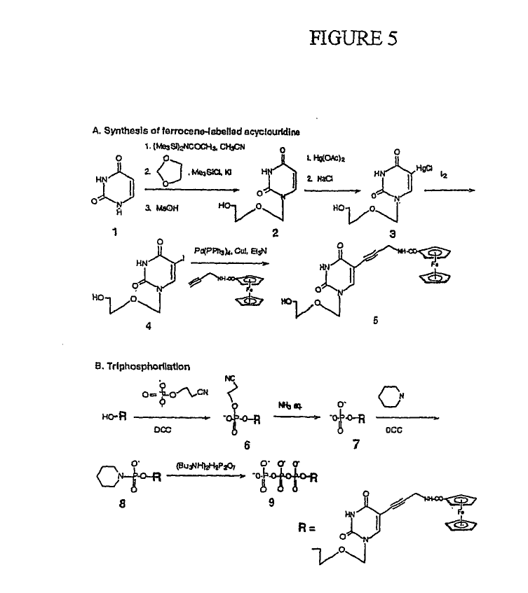

A first alternative synthesis of ferrocene-labelled acyclonucleoside triphosphate derivatives took place using the reaction outlined in FIG. 5.

### Example 10

### Introduction of a Vinylferrocene Residue into a Nucleotide—A Second Alternative Synthetic Route

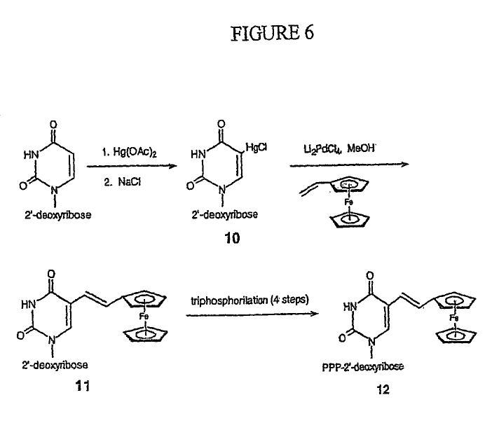

Introduction of a vinylferrocene residue into a nucleotide will allow full conjugation between the nucleobase and the ferrocene residue, which may be beneficial for electron transfer between ferrocene, the DNA π-stack and an electrode. Synthesis of a vinylferrocene-containing derivative of dUTP is set out in FIG. 6.

10 μmol of Hg-dUTP was dissolved in 1 mL of water, and 50 μmol of dry vinylferrocene and 1 mL of 0.1 M Li₂PdCl₄ in methanol were added to this solution. The dark-blue mixture was stirred in darkness for 12 h. The colour gradually disappears, and black precipitate of Pd forms in the solution. The precipitate was filtered off, and the solution loaded onto DEAE-cellulose column. The column was washed with 50% methanol, and then with a gradient of 0-0.4 M triethylammonium bicarbonate (pH 7.0) in 30% ethanol. Final purification was achieved by reverse-phase HPLC on a Zorbax column (1-25 cm) in a 0-30% gradient of acetonitrile in 50 mM LiClO₄. Product-containing fractions were evaporated, dissolved in a minimum volume of water, and precipitated by addition of 10 volumes of 2% LiCl₄ in acetone. The precipitate was dried in air. Yield 5%.

### Example 11

### Synthesis of 3-Ferrocenecarboxamidopropnyl-1

1 eq. of ferrocenecarboxylic acid, 1.2 eq. of DCC, 1.2 eq. of HOBt, and 2 eq. of propargylamine were dissolved in dichloromethane and stirred overnight. The precipitate of dicyclohexylurea was filtered off and washed with CH₂Cl₂. Combined liquids were evaporated and applied on silica gel column. The product was purified in the gradient of 0-10% MeOH in CH2Cl2 and dried in vacuo. Yield 92%.

### Example 12

### Synthesis of 5-(3-Ferrocenecarboxamidopropynyl-1)-acyclouridine

1 eq. of 5-iodoacyclouridine, 1 eq. of 3-ferrocenecarboxamidopropynyl-1, 1 eq. of triethylamine, 0.1 eq. of Pd(PPh₃)₄, and 0.2 eq. of CuI were mixed in anhydrous DMF and stirred at room temperature for 4 h. DMF was removed in vacuo, and the residue was applied to a silica column. The product was purified in the gradient of 0-20% MeOH in CH₂Cl₂ (product was eluted at 15% of MeOH). Organic solvents were removed in vacuo. Yield 20%.

### Example 13

### Synthesis of 5-(3-Ferrocenecarboxamidopropnyl-1)-acyclouridine 5′-triphosphate

5-(3-Ferrocenecarboxamidopropynyl-1)-acyclouridine from Example 12 was evaporated 3 times with anhydrous pyridine, dissolved in 0.5 mL of triethylphosphate and cooled on ice. 1 eq. of POCl₃ was added to the solution After 3 min of incubation on ice, the mixture of 10 eq. 0.5 M tributylammonium pyrophosphate in anhydrous DMF, 1 mL DMF and 0.24 mL tributylamine was added and stirred for 1 min. The reaction was stopped by addition of 10 mL of 1 M TEAB (pH 7.0). The mixture was evaporated, dissolved in 20% aqueous EtOH and separated on DEAE-cellulose in a gradient of 0-0.4 M TEAB in 20% ethanol. Final purification was achieved by reverse-phase HPLC on a Zorbax column in the gradient of 0-30% acetonitrile in 50 mM LiClO₄. Product-containing fractions were evaporated, dissolved in minimum volume of water, and precipitated by addition of 10 volumes of 2% LiClO₄ in acetone. The precipitate was dried on air. Yield 3%. The yield is very low due to the side reaction of POCl₃ with the ferrocene residue.

### INDUSTRIAL APPLICATION

Conjugation of ferrocene and other redox-active moieties with nucleoside triphosphates enables the broad expansion and diffusion of electrochemical methodologies in molecular biology and genetic analysis. Enzymatic redox labelling of nucleic acids has a range of applications in DNA sequencing, mRNA expression analysis and genotyping.

### REFERENCES

Langer, P. R., Waldrop, A. A. and Ward, D. C. (1981) Enzymatic synthesis of biotin-labelled polynucleotides: novel nucleic acid affinity probes. Proc. Natl. Acac. Sci. USA 78: 6633-6637.

Sambrook, J., Fritsch, E. F. and Maniatis, T. (1989). Molecular Cloning. A Laboratory Manual. Cold Spring Harbor, N.Y., Cold Spring Harbor Laborator Press.

Johnston, D. H. (1995) Electrochemical measurement of the solvent accessibility of nucleobases using electron transfer between DNA and metal complexes. J. Am. Chem. Soc. 117: 8933-8938.

Shigenaga, M. K. (1990) In vivo oxidative DNA damage: measurement of 8-hydroxy-2′-deoxyguanosine in DNA and urine by high-performance liquid chromatography with electrochemical detection. Methods Enzymol. 186: 521-530.

Takenaka, S., Uto, Y., Kondo, H., Ihara, T. and Takagi, M. (1994) Electrochemically active DNA probes: detection of target DNA sequences at femtomole level by high-performance liquid chromatography with electrochemical detection. Anal. Biochem. 218: 436-443.

Umek, R. M., Lin, S. W., Vielnetter, J., Terbrueggen, R. H., Irvine, B., Yu, C. J., Kayyem, J. F., Yowanto, H., Blackburn, G. F., Farkas, D. H. and Chen, Y.-P. (2001) Electronic detection of nucleic acids. A versatile platform for molecular diagnostics. J. Mol. Diag. 3(2):74-84.

Woolley, A. T., Lao, K Q., Glazer, A. N. and Mathies, R. A. (1998) Capillary electrophoresis chips with integrated electrochemical detection. Anal. Chem. 70(4): 684-688.

The foregoing describes embodiments of the present invention and modifications, obvious to those skilled in the art can be made thereto, without departing from the scope of the present invention.

1. A modified nucleoside analogue having the formula (I):
P-S-B-L-R
where:
P is a 5′ triphosphate or analogue or derivative thereof;
S is a substituted or unsubstituted five- or six-membered sugar, sugar analogue or acyclo sugar analogue, but excluding a dideoxy-sugar;
B is a substituted or unsubstituted nitrogenous base or base analogue or derivative thereof;
L is a linker group; and
R is a substituted or unsubstituted metallocene moiety or substituted or unsubstituted metal complex or substituted or unsubstituted redox-active organic moiety.

2. The modified nucleoside analogue as claimed in claim 1 wherein P is an enzyme-compatible triphosphate moiety.

3. The modified nucleoside analogue as claimed in claim 2 wherein P is selected from the group consisting of triphosphate, α-thiotriphosphate, β-thiotriphosphate, γ-thiotriphosphate, α-dithiotriphosphate, or β,γ-methylenetriphosphate.

4. The modified nucleoside analogue as claimed in claim 1 wherein group S is substituted or unsubstituted ribose, 2′-deoxyribose, 3′-fluoro-2′-deoxyribose 3′-amino-2′-deoxyribose, a bicyclic “locked” LNA sugar selected from 2′-0,4′-C-methylene-, 2′-C,4′-C-ethylene- or 2′-0,4′C-ethylene-bridged furanose, or an acyclo moiety comprising a 2-hydroxyethoxymethyl group or analogue thereof.

5. The modified nucleoside analogue as claimed in claim 4 wherein group S is substituted with substitutes selected from one or more of fluoro, amino, hydroxyl, methyl or methoxy groups.

6. The modified nucleoside analogue as claimed in claim 4 wherein group S is unsubstituted.

7. The modified nucleoside analogue as claimed in claim 1 wherein B is a substituted or unsubstituted purine or pyrimidine or other nucleobase or nucleobase analogue.

8. The modified nucleoside analogue as claimed in claim 7 wherein B is adenine, guanine, cytosine, uracil, or thymine, inosine or a derivative of an adenine, guanine, cytosine, uracil, thymine or inosine.

9. The modified nucleoside analogue as claimed in claim 8 wherein B is a 7-deaza variant of adenine or guanine.

10. The modified nucleoside analogue as claimed in claim 8 wherein said derivative includes at least one this group.

11. The modified nucleoside analogue as claimed in claim 1 wherein L is or contains a saturated or unsaturated aliphatic chain, with or without cyclic groups or an amine or a carboxyl or an amide.

12. The modified nucleoside analogue as claimed in claim 11 wherein L is substituted with fluoro, ether or hydroxy substituents.

13. The modified nucleoside analogue as claimed in claim 11 wherein L is of 1-24 bonds in contour length.

14. The modified nucleoside analogue as claimed in claim 13 wherein L is of 3-12 bonds in length.

15. The modified nucleoside analogue as claimed in claim 11 wherein L is selected from propenyl or propargyl derivatives.

16. The modified nucleoside analogue as claimed in claim 1 wherein R is a substituted or unsubstituted metallocene, a substituted or unsubstituted metal complex or an organic redox moiety and wherein substituents are selected from one or more of the groups fluoro, bromo, chloro, methyl, ethyl, hydroxy, hydroxymethyl, hydroxyethyl, methoxy, ethoxy, acetyl, cyano, thiocyano, amino, nitro, vinyl, amido, methylamido, and dimethylamido.

17. The modified nucleoside analogue as claimed in claim 16 wherein R is a metallocene having a redox potential in the range of −1.0 to +1.0 V vs. Standard Hydrogen Electrode (SHE).

18. The modified nucleoside analogue as claimed in claim 1 wherein R is ferrocene.

19. The modified nucleoside analogues as claimed in claim 1 wherein R is a quinone or quinone-containing moiety.

20. The modified nucleoside analogue as claimed in claim 19 wherein said quinone or quinone-containing moiety is selected from anthraquinones and substituted anthraquinones.

21. The modified nucleoside analogue as claimed in claim 16 wherein R is a metal complex exhibiting reversible electron transfer with Eo in the range −1 V to +1 V vs standard hydrogen electrode (SHE).

22. The modified nucleoside analogue as claimed in claim 1 wherein B is selected from substituted or unsubstituted cytosine, uracil or thymine and L is joined to the CS carbon of the cytosine, uracil or thymine.

23. The modified nucleoside analogue as claimed in claim 1 wherein B is selected from substituted or unsubstituted adenine or guanine or a 7-deaza-derivative of adenine or guanine in which the N7 is replaced by a C7 and L is joined to the C8 carbon or to the C7 carbon.

24. A method of synthesising a the modified nucleoside analogue of claim 1 comprising reacting a nucleoside or nucleotide precursor with a metallocene, metal complex or organic redox moiety precursor so as to form a link between the nucleos(t)ide analogue and the metallocene, metal complex or organic redox moiety.

25. A method as claimed in claim 24 further comprising the step of subsequently incorporating a 5′ triphosphate or derivative thereof wherein the starting nucleoside for nucleotide precursor does not include such a triphosphate or triphosphate derivative.

26. A method-as claimed in claim 25 wherein the link between the nucleos(t)ide precursor and the metallocene, metal complex or organic redox moiety is formed by a condensation reaction and the method further includes the step of adding a condensing agent.

27. A method as claimed in claim 24 wherein the link between the nucleos(t)ide analogue and the metallocene, metal chelate or organic redox moiety is formed by a displacement reaction.

28. A method as claimed in claim 24 wherein the method comprises reacting a nuceloside or nucleotide precursor with a metallocene precursor in the presence of a condensing agent so as to form a link between the nucleoside analogue and the metallocene or derivative thereof.

29. A method as claimed in claim 24 wherein the nucleotide precursor is selected from uridine 5′-triphosphate, cytidine 5′-triphosphate, adenosine 5′-triphosphate, guanosine 5′-triphosphate, 2′ deoxyadenosine 5′-triphosphate, 2′deoxyguanosine 5′-triphosphate, 2′ deoxythymidine 5′-triphosphate, 2′-deoxyuridine 5′-triphosphate, 2′ deoxycytidine 5′-triphosphate, 5-aminoallyl-uridine-5′-triphosphate, 5-aminopropargyl-uridine-5′-triphosphate, 5′-aminoallyl-cytidine-5′-triphosphate, 5-aminopropargyl-cytidine-5′-triphosphate, 7-aminopropargyl-deazaadenosine-5′-triphosphate, 7-aminopropargyl-deazaguanosine-5′-triphosphate, 5-aminoallyl-2′-deoxyuridine-5′-triphosphate, 5-aminopropargyl-2′-deoxyuridine-5′-triphosphate, 5-aminoallyl-2′ deoxycytidine-5′-triphosphate, 5-aminopropargyl-2′-deoxycytidine-5′-triphosphate, 7-aminopropargyl-7-deaza-2′ -deoxyadenosine-5′-triphosphate, 7-aminopropargyl-7-deaza-2′-deoxyguanosine-5′-triphosphate, 5-aminopropargyl-acyclouridine-triphosphate, 5-aminopropargyl-acyclocytidine-triphosphate, 7-aminopropargyl-acyclodeazaadenosine-triphosphate, or 7-aminopropargle-acyclodeazaguanosinne-triphoshate.

30. A method as claimed in claim 24 wherein the metallocene precursor is a carboxylic acid.

31. A method as claimed in claim 30 wherein the metallocene precursor is ferrocenecarboxylic acid or forroceneacetic acid or derivative thereof.

32. A method as claimed in claim 26 wherein the condensing agent is selected from any one of a carbodiimide, for example dicyclohoxylcarbodiimide, uronium compounds, activated ethers and other compounds employed in the formation of amide bonds.

33. A method as claimed in claim 26 wherein the condensing agent is 0-benzotriazol-1-yl-N,N,N′,N′-tetramethyluronium hexafluorophosphate (HBTU).

34. An oligo- or poly-nucleotide probe, primer or enzymatic reaction product comprising at least one residue of a nucleoside analogue according to claim 1.

35. An oligo- or poly-nucleotide probe, primer or enzymatic reaction product as claimed in claim 34 wherein the at least one residue of the nucleoside analogue comprises at least one residue of a metallocene nucleoside analogue.

36. A method of nucleotide chain incorporation, the method comprising reacting a template nucleotide chain with a modified nucleoside analogue of claim 1 in the presence of a processive nucleotidyl transferase or polymerase.

37. A method of nucleotide chain extension, the method comprising reacting a nucleotide chain with a modified nucleoside analogue of claim 1 in the presence of a non-processive nucleotidyl transferase such a terminal transferase or poly(A) polymerase.

38. A method of electrochemical detection of DNA, RNA, DNA/RNA chimers or nucleic acid analogues, the method comprising incorporating the modified nucleoside analogue of claim 1 into a nucleic acid chain and detecting the analogue on the basis of its redox potential.

39. A method of electrochemical detection of DNA, RNA, DNA/RNA chimers or nucleic acid analogues, the method comprising incorporating two or more different modified nucleoside analogues of claim 1 into the same or different nucleic acid chains, and detecting the modified nucleoside analogues on the basis of their different redox potentials.

40. A kit comprising, and the analogues of claim 1 and at least one nucleotidyl transferase enzyme(s).

41. A kit as claimed in claim 40 further comprising one or more of an appropriate unlabelled nucleotide mix, an optimised reaction buffer, control template and primer so that the user may determine the efficiency of DNA synthesis.

42. The modified nucleoside analogue as claimed in claim 21 said metal complex is selected from chelates and cryptates of transition metals including iron, copper, cobalt, ruthenium, rhodium, osmium complexed with bi-, tri-, tetra-, hexa- or octadentate ligands, said metal complex.

43. The modified nucleoside as claimed in claim 42 wherein said metal complex include one or more ligands selected from tridentate N-donor ligands, such as terpyridine (terpy), bis(benzimidazolyl)pyridines (bzimpy) and bis(pyrazolyl)pyridines (bpp), as well as mixed O,N,O donor ligands such as pyridinedicarboxylic acid (dipic).


SEQUENCE LISTING (6 sequences)

SEQ ID NO: 1 - DNA, 18 bp, Artificial
  18-mer primer
caacgtccgagcagtaca

SEQ ID NO: 2 - DNA, 40 bp, Artificial
  40-mer template
aagctccttagtctgtcaatgtactgctcggacgttgcga

SEQ ID NO: 3 - DNA, 25 bp, Artificial
  25-mer template
gctgatggagctcggtgttttgctt

SEQ ID NO: 4 - DNA, 31 bp, Artificial
  31-mer template
tatataagcttcatagaccagttacctcatg

SEQ ID NO: 5 - DNA, 40 bp, Artificial
  40-mer template
aagctccttagtctgtcaatgtactgctcggacgttgcta

SEQ ID NO: 6 - DNA, 18 bp, Artificial
  18-mer primer
caacgtccgagcagtaca